(19) United States
(12) Patent Application Publication
(10) Pub. No.: US 2017/0164942 A1
(43) Pub. Date: Jun. 15, 2017

(54) Holding Block For Multiple Catheter Needles

(71) Applicant: HeartStitch, Inc., Fountain Valley, CA (US)

(72) Inventors: Anthony Nobles, Fountain Valley, CA (US); Linda Weichenhain, Zwickau (DE)

(21) Appl. No.: 15/133,815

(22) Filed: Apr. 20, 2016

Related U.S. Application Data
(60) Provisional application No. 62/267,827, filed on Dec. 15, 2015.

(51) Int. Cl. A61B 17/04; A61B 17/062

(52) U.S. Cl. CPC A61B 17/0469; A61B 17/0482; A61B 17/0625; A61B 2017/0472

(57) ABSTRACT

A suture device with a holding block, and methods of controlling relative positions of ends of multiple suture needles in the suture device, are presented. The suture device has two or more suture needles, and a guide wire, all of which are coupled to the holding block. Suture needles are affixed at the holding block at two distinct contact regions and the guide wire is disposed at the holding block in a slidable relationship. By affixing two needles at the holding block, the relative positions of ends of suture needles can be maintained in the suture device, so that two needles can capture sutures simultaneously, even when two needles are curved in opposite directions.

This application claims the benefit of priority to U.S. Provisional Patent Application No. 62/267,827, filed Dec. 15, 2015, which is incorporated by reference herein in its entirety.

### FIELD OF THE INVENTION

The field of the invention is methods and devices for holding multiple catheter suture needles.

### BACKGROUND

The background description includes information that may be useful in understanding the present invention. It is not an admission that any of the information provided herein is prior art or relevant to the presently claimed invention, or that any publication specifically or implicitly referenced is prior art.

Health practitioners frequently use sutures to close various openings such as cuts, punctures, and incisions in various places in the human body. Depending on types and sizes of tissues to be sutured, and their locations, various different types of needles, suture threads, and suture devices can be used in the suturing process. Some suture devices have multiple needles. Yet, when two or more needles are located in one suturing device, and the suturing device is used for endoscopic suturing, it can be problematic to maintain the relative effective length of the various needles at the area being sutured.

Some efforts have been made to solve the problem by prepositioning the needle(s) on a pivot at the suturing end of the catheter or trocar. See, for example, U.S. Pat. No. 5,364,408 to Gordon. Unfortunately, such prepositioning can overly restrict the throw distance or radius of the needle suturing path.

Others have solved the problem by independently positioning and using the various needles. See for another example, U.S. Pat. No. 5,993,466 to Yoon and U.S. Patent Application No. 2013/0066340 to Pantages. Of course, such independent control can put unnecessary operational burden on the physician/operator/user.

Thus, none of prior art teaches any mechanism that can direct multiple needles along a length of a catheter or trocar, while maintaining a desired relative length of the needles at the suture site, without separately controlling travel of the needles through the catheter or trocar.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Thus, there is still a need for improved methods and devices for directing multiple needles along a length of a catheter or trocar, while maintaining a desired relative length of the needles at the suture site, without separately controlling travel of the needles through the catheter or trocar.

### SUMMARY OF THE INVENTION

The inventive subject matter provides systems and methods for directing multiple needles along a length of a catheter or trocar, while maintaining a desired relative length of the needles at the suture site, without separately controlling travel of the needles through the catheter or trocar.

One aspect of the invention includes a suturing device that includes more than one suture needle, a guide wire, and a holding block affixed to the suture needles. The holding block is preferably disposed in slidable relationship with the guide wire, but in fixed (non-sliding) relationship with each of the needles. The fixed relationship(s) between holding block and needles can be established in any suitable manner, including fixing the needle within a passageway, hole, ridge, fold, or portion of a surface of the holding block. Preferably, less than 10% of the length(s) of the needles are directly held by the holding block.

Another aspect of the invention includes methods of controlling relative positions of distal (away from the physician/operator/user) ends of multiple suture needles, when the needles are deployed through a length of a catheter or trocar. Contemplated methods include steps of affixing the suture needles to a holding block, placing the holding block with the suture needles inside a catheter or trocar, and in a preferred embodiment, sliding a guide wire through the holding block.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a perspective view of an embodiment of a holding block for multiple catheter needles.

FIG. 2A is a perspective view of the holding block of FIG. 1, assembled with catheter needles and a guide wire, as the needles might be straight within a catheter or curved trocar.

FIG. 2B is a perspective view of the holding block of FIG. 1, assembled with catheter needles, as the needles might be curved within a catheter or curved trocar.

FIG. 3A is a sectional view of a portion of a suturing device, shown without a holding block.

FIG. 3B is a cross-sectional view of a portion of the suturing device of FIG. 3A, taken at the plane P, shown with a holding block.

### DETAILED DESCRIPTION

The inventive subject matter provides a suturing device having a holding block configured to hold multiple suture needles and optionally a guide wire. In addition, the inventive subject matter provides methods for controlling relative positions of ends of multiple suture needles, upon utilization of the needles inside a catheter or trocar.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities or ranges, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

One aspect of the inventive subject matter includes a suturing device. The suturing device includes at least two or more suture needles and a guide wire. The suturing device also includes a holding block. In a preferred embodiment, the holding block is removably coupled with a portion of the suturing device. In other embodiments, the holding block is affixed to a portion of the suturing device (e.g., permanently attached).

For purposes of the present application, the term “suture needle” means both a length of metal or other material intended to pass through a tissue, along with an extension portion that can be used to direct translational or other motion of the working end of the needle. Thus, as used herein, a suture needle would typically be longer than the lumen of a catheter or trocar in which the needle is to be utilized. The length of suture needles and guide wires can vary depending on the use of the suture device. For endoscopic suturing via vascular structures, it is preferred that lengths of suture needles and guide wires are at least 10 cm, preferably at least 20 cm, and in at least some embodiments, more preferably at least 30 cm.

All suitable suture needles are contemplated, including especially needles having a hook that can be used in for throw-and-catch suturing.

Preferably, suture needles and guide wires comprises metal alloy materials (e.g., steel, solder, brass, nickel titanium, etc.) or metal materials (e.g., aluminum, copper, platinum, etc). Yet, it is also contemplated that suture needles or guide wires can comprise other types of materials such as plastic materials (e.g., polypropylene, polyethylene, nylon, PVC or PTFE) or glass fibers.

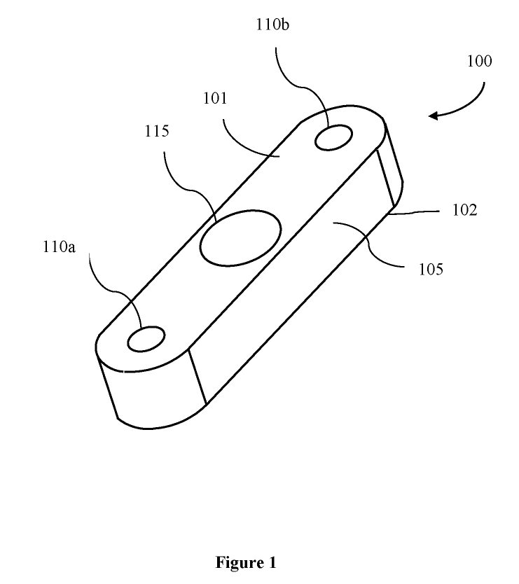

FIG. 1 shows one embodiment of a holding block 100, having a distal surface 101, proximal surface 102, and a lateral surface 105. The holding block 100 can have any suitable shape, as long as it can be fitted within the suture device. For example, the holding block 100 can have a tubular shape, a twisted tubular shape, an elliptical tubular shape, a cuboid shape, a triangular prism shape, etc. In some embodiments, a portion of a holding block 100 includes a protrusion (e.g., overhang, bulge, etc.) or a depression (e.g., indent, recess, etc) to facilitate insertion of the holding block 100 into a suture device.

It is contemplated that holding block 100 can be made of any suitable material that is compatible with a suturing device with which it engages. For example, holding block 100 can comprise metal alloy (e.g., steel, solder, brass, nickel titanium, etc.), plastic materials (e.g., polypropylene, polyethylene, nylon, PVC or PTFE), metal materials (e.g., aluminum, copper, platinum, etc), or fiber (e.g., cotton, synthetic fiber, glass fiber, etc.). In a preferred embodiment, at least a portion of the holding block 100 comprises nickel titanium (nitinol) such that the holding block 100 can have shape memory.

The holding block 100 further includes at least two contact regions 110a, 110b for affixing suture needles. As illustrated in FIG. 1, contact regions 110a, 110b are apertures or holes that extend between the proximal surface 102 and the distal surface 101. However, it is also contemplated that the two or more contact regions could be in any suitable shapes to hold suture needles. For example, in some embodiments, the contact regions could be ridges, folds, or portions of a surface. In some embodiments, the contact regions could be located on a portion of lateral surface 105 of the holding block 100.

Preferred methods of permanently affixing suture needles to contact regions 110a, 110b include gluing, sealing, and soldering. In other embodiments, the contact regions 110a, 110b could temporarily immobilize the suture needles, for example by mechanically holding between two arms using crimping, etc. The key requirement is that the suture needles cannot appreciably slide relative to the holding block 100 during ordinary use.

The contact regions are preferably rather short, preferably less than 30%, more preferably less than 20%, still more preferably less than 10% of a length of a suture needle engaged with the contact regions. As used herein, the length of a contact region is defined as a distance between one end of the contact region closest to the proximal surface 102 and the other end of the contact region closest to the distal surface 101 of the holding block 100.

The holding block 100 further includes a path 115 for a guide wire. In a preferred embodiment, the path 115 is an aperture or a hole disposed in the holding block 100, which allows the guide wire to pass between the proximal surface 102 and the distal surface 101 of the holding block 100. In this embodiment, it is especially preferred that a diameter of the path is slightly larger than a diameter of the guide wire, such that the guide wire can be disposed within the path in a slidable relationship with each other.

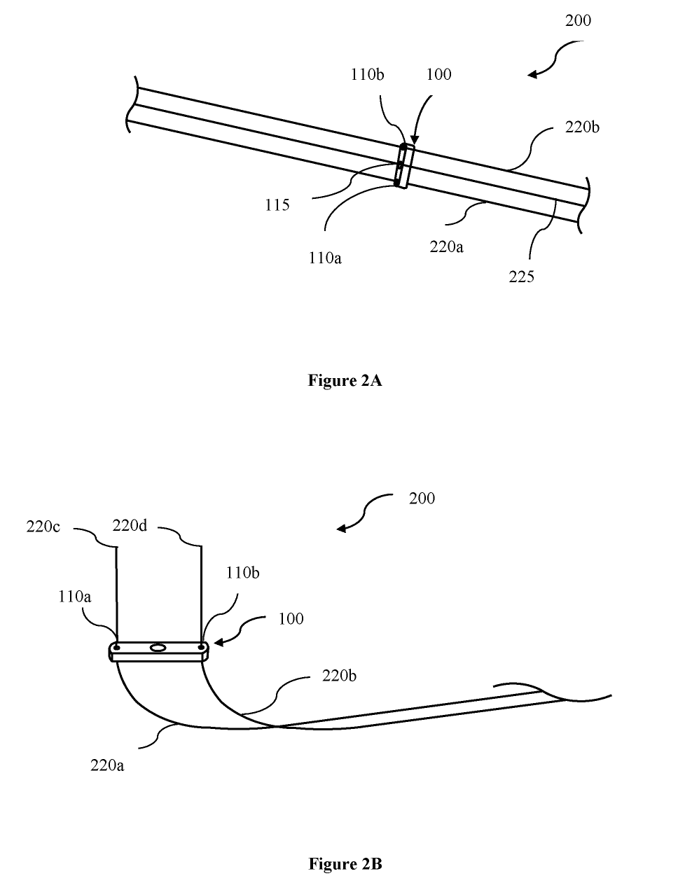

FIGS. 2A and 2B show perspective views of an embodiment of a holding block 200, assembled with suture needles 220a, 220b, and a guide wire 225, where the needles 220a, 220b and guide wire 225 are straight (FIG. 2A) or curved (FIG. 2B), respectively. Suture needles 220a, 220b are held (e.g., affixed) at the contact regions 110a, 110b. The guide wire 225 is slidably engaged within the path 115.

When the holding block 200 with suture needles 220a, 220b and guide wire 225 are straight (i.e., without significant bending), the relative end positions (not shown) of suture needles 220a, 220b can be maintained to pick up sutures at two separate locations simultaneously. However, if the suture needles 220a, 220b are curved (whether in an opposite direction or in the same direction), it is likely that relative end positions of the suture needles 220a, 220b cannot be maintained (e.g., one end locates more proximally than the other, etc.) and the sutures cannot readily be picked up simultaneously. As shown in FIG. 2B, the relative end positions 220c, 220d of the suture needles 220a, 220b are maintained by virtue of the two suture needles 220a, 220b being affixed to holding block 200, even though the suture needles 220a, 220b are curved.

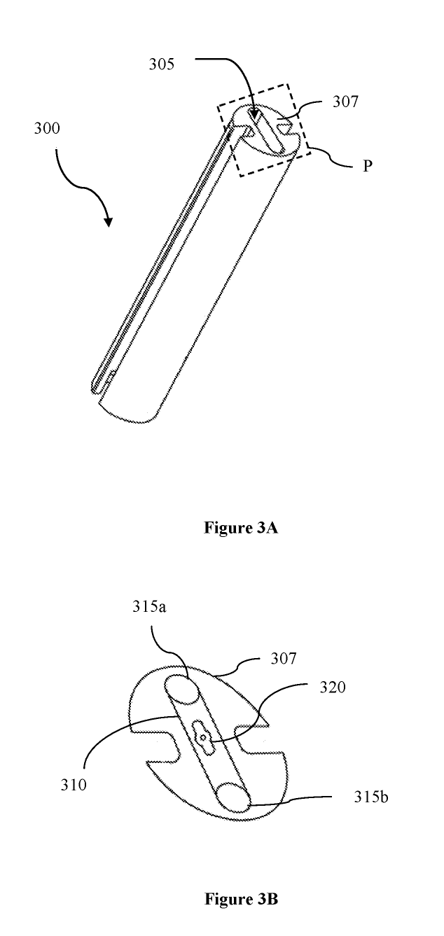

FIG. 3A shows a sectional view of an embodiment of a suturing device 300. FIG. 3B shows a cross sectional view (corresponding to plane P in FIG. 3A) of the same suturing device 300, but here holding block 310 has been positioned within the suturing device 300. In this embodiment, the suturing device has a hollow space 305 within the body 307, in which the holding block 310 can be slidably inserted. It is contemplated that the holding block 310 can be removed and replaced with another if the suture needles are damaged or need to be replaced. The holding block 310 has two contact regions 315a, 315b where suture needles can be affixed, and a path 320 through which a guide wire can pass. Although the preferred embodiments include two contact regions for two suture needles, it is also contemplated that a different holding block could have more than two contact regions, to facilitate concurrent use of more than two suture needles.

Another aspect of the invention includes methods of controlling relative positions of the ends of two or more suture needles, upon utilization of the needles inside a catheter or trocar. In such methods, at least two suture needles are affixed to a holding block at two distinct contact regions of the holding block, and the holding block with the suture needles are placed inside the catheter, trocar, or other suture device. In a preferred embodiment, the method further includes a step of slidably engaging a guide wire within a path through the holding block. Preferably, the path is distinct from the contact regions. More preferably, the path is located between two contact regions.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

This listing of claims represents the current version of claims before the Office:

1. A suturing device, comprising:
first and second suture needles;
a guide wire; and
a holding block affixed to the first and second suture needles, and disposed in slidable relationship with the guide wire.

2. The device of claim 1, wherein the first needle comprises a metal alloy.

3. The device of claim 1, wherein the first needle has a length of at least 20 cm.

4. The device of claim 1, wherein the holding block comprises a metal alloy.

5. The device of claim 3, wherein the metal alloy is nickel titanium.

6. The device of claim 1, wherein the first and second suture needles are affixed at first and second contact regions, and a length of the first contact region is less than 10% of a length of the first needle.

7. The device of claim 6, wherein the first contact region is a hole, a ridge, a fold, or a portion of a surface.

8. The device of claim 6, wherein the first suture needle are glued at the first contact region.

9. The device of claim 1, wherein the guide wire is located between the first and second suture needles.

10. A method of controlling relative positions of ends of first and second suture needles upon utilization of the needles inside a catheter, comprising:
affixing the first and second suture needles to a holding block;
placing the holding block with the first and second suture needles inside the catheter; and
when the needles are deployed inside a length of the catheter, directing the needles by curving the needles in a same direction within the catheter, and maintaining a desired relative length of the needles at a suture site, distal to the holding block, without independently controlling the respective length of the needles proximal to the holding block.

11. The method of claim 10, further comprising sliding a guide wire through the holding block.

12. The method of claim 11, wherein the guide wire is located between the first and second suture needles.

13. The method of claim 10, wherein the holding block comprises a metal alloy.

14. The method of claim 13, wherein affixing is soldering a portion of the first needle with a portion of the holding block.

15. The method of claim 13, wherein the metal alloy is nickel titanium.

16. The method of claim 10, wherein the first and second suture needles are affixed at first and second contact regions, and a length of the first contact region is less than 10% of a length of the first needle.

17. The method of claim 16, wherein the first contact region is a hole, a ridge, a fold, or a portion of a surface.

18. The method claim 16, wherein affixing is gluing or crimping.
